(19) United States
(12) Patent Application Publication
(10) Pub. No.: US 2011/0263606 A1
(43) Pub. Date: Oct. 27, 2011

(54) SOLID ORAL DOSAGE FORMS COMPRISING TADALAFIL

(76) Inventors: Horst Zerbe, Hudson (CA); Nadine Paiement, Ville St. Laurent (CA); Angela Angusti, Montreal (CA); Cormac Long, LaSalle (CA)

(21) Appl. No.: 13/079,348

(22) Filed: Apr. 4, 2011

Related U.S. Application Data
(60) Provisional application No. 61/327,969, filed on Apr. 26, 2010.

(51) Int. Cl. A61K 31/4985; A61P 11/00; B29D 7/01; A61P 9/12

(52) U.S. Cl. 514/250; 264/212

(57) ABSTRACT

Improved pharmaceutical solid oral dosage forms for the buccal and/or sublingual delivery of Tadalafil. The improved delivery systems for solubilizing and stabilizing pharmaceutically active ingredients exhibit enhanced methods of preparation by the use improved solubilization systems which can maintain the Tadalafil in a buccal and/or sublingual oral dosage form or a polymeric film matrix that provides improved bioavailability and/or absorption of Tadalafil.

### CROSS REFERENCE TO RELATED APPLICATION

This application claims benefit under 35 USC §119(e) of provisional application Ser. No. 61/327,969, filed Apr. 26, 2010, entitled “METHODS FOR MAKING IMPROVED SOLID ORAL DOSAGE FORMS COMPRISING TADALAFIL,” the entire contents of which are incorporated herein by reference.

### FIELD OF THE INVENTION

This invention relates to an improved process for the preparation of solid oral pharmaceutical dosage forms comprising Tadalafil and preferably for buccal and/or sublingual oral film dosage forms comprising Tadalafil demonstrating improved bioavailability.

### BACKGROUND OF THE INVENTION

Tadalafil has been used for the treatment of male erectile dysfunction and has the chemical name (6R-trans)-6-(1,3-benzodioxol-5-yl)-2,3,6,7,12,12a-hexahydro-2-methyl-pyrazino[1′,2′:1,6]pyrido[3,4-b]indole-1,4-dione. Tadalafil is a solid that is understood to be practically insoluble in water and only very slightly soluble in some organic solvents. The extremely limited solubility of Tadalafil poses many major difficulties and challenges when formulating a dosage form that demonstrates acceptable bioavailability.

A pharmaceutically employed oral film is formulated to exhibit instant hydration followed by a rapid dissolution/disintegration upon administration into the oral cavity. Upon administration and dissolution, the patient will not feel any discomfort during and/or immediately after its dissolution. The disintegration time can be varied through the suitable adjustment of the composition and physical properties of the matrix. Film forming polymers of common pharmaceutical use are water-soluble or water dispersible polymers that conform to the required properties, including, but not limited to, film instant hydration potential, mucoadhesion and solubility over time. Examples of film forming polymers include cellulose derivatives, polyvinyl alcohol, polyvinyl pyrrolidone, starches, polyacrylates, gums (xanthane gum, arabic gum, guar gum, etc.) and/or mixtures thereof. Film forming polymers may be used in combinations chosen based on the desired characteristics of the delivery form (e.g., rapid disintegration, higher mucoadhesion, longer residence time, etc.).

The prior art discloses several methods to improve the bioavailability of poorly soluble drugs, for example, modifying the drug itself. The physical properties of an active ingredient can be altered using various techniques to optimize the rate at which the drug is dissolved. The most commonly employed of these techniques and the one most relevant to the present invention is particle size reduction. Particle size reduction has been a non specific formulation approach that can be applied to almost any drug to enhance solubility. The increase in surface area results in a significant increase in surface energy leading to greater solubilization.

There are many challenges associated with the manufacture of oral film dosage forms ranging from brittleness, tackiness, the hygroscopic nature and potential lack of homogeneity within the dosage form. Ideal physical characteristics of the oral film include dosage uniformity throughout the dosage form, adequate flexibility and tensile strength to facilitate processing, handling, and packaging of the film in a consumer-friendly form. Attaining ideal conditions for one characteristic usually comes at the expense of other, often equally important, properties, resulting in a necessary compromise in various properties to achieve a working film dosage form.

The preparation of an oral film dosage form requires that the final blend has a critical lower viscosity limit as this greatly affects the film casting potential. This is due to the fact that the final blend is transferred onto a surface of a suitable carrier material upon which the blend is cast and dried to form a film. Optimal viscosity ranges from 1000 centipoise to 90,000 centipoise. If the viscosity of the blend is too low there is a significant risk of not facilitating the formation of film after coating the blend on the carrier. The mixtures may not be homogeneous, and the drying resistance of a film tends to be low. In order to produce a solid oral film dosage form comprising Tadalafil and demonstrating improved bioavailability of the Tadalafil, a blend must be produced that provides sufficient solubilization of the Tadalafil as to produce a blend containing a film forming polymer capable of producing a solid oral film dosage form, and with sufficient viscosity as to be coated onto a carrier system and successfully form a solid oral film dosage form with acceptable dimensions and drug loading. If the solubility of the Tadalafil is too low the solvent required to dissolve the Tadalafil would make it extremely difficult or impossible to achieve optimal viscosity, acceptable dimensions, and adequate drug loading. Due to the nature of the solid oral film dosage form manufacturing process, the low vapor pressure of preferred Tadalafil liquid solvents, regulatory body (e.g., the United States Food and Drug Administration) imposed residual solvent limits, and the undesirability of heating a system to well above room temperature, preferred systems include solvents with reasonably high residual limits and low boiling points. The prior art discloses many solvent systems for dissolving Tadalafil, but does not fully address the difficulty associated with achieving the desired improved solubility of Tadalafil when preparing a pharmaceutical film capable of achieving improved bioavailability of the Tadalafil upon buccal and/or sublingual oral administration.

### SUMMARY OF THE INVENTION

In accordance with certain aspects of the invention, improved solubilization and stabilization of Tadalafil are achieved for a solid film dosage form that exhibits enhanced bioavailability and/or absorption of Tadalafil when administered orally.

The invention is generally directed to improved pharmaceutical oral dosage forms comprising Tadalafil, at least one Tadalafil solubility enhancer, and optionally including one or more plasticizers, penetration enhancing substances, surfactants, sweetening agents, flavors, flavor enhancers, antioxidants, starches, and/or colorants, that provide improved characteristics such as those relating to disintegration, and drug absorption, and methods for making same.

### DETAILED DESCRIPTION OF PREFERRED EMBODIMENT

Unless otherwise indicated, terms in this specification are intended to have their ordinary meaning in the relevant art.

In accordance with certain embodiments of the invention, an improved process for the manufacture of solid oral film dosage forms comprising Tadalafil is provided.

Among other things, there is disclosed an improved mechanisms to achieve a desired release profile for Tadalafil. While a rapid solubilization of the Tadalafil is preferred, various desired solubilization profiles (i.e., plots of the quantity or quantities of Tadalafil absorbed by a liquid medium or mediums at particular time points) can be achieved by adjusting the properties of and procedures for producing the film dosage form. The increase in solubility of Tadalafil is due to a combination of an increase in the surface energy of the active particles and the stabilization of such. Factors which contribute to the improved bioavailability of the active include a surprising and unforeseeable ability of the invention to provide a process that demonstrates a remarkably improved degree of solubilization of Tadalafil and to such an extent as to be capable of producing a solid oral film dosage form comprising Tadalafil with acceptable dimensions and drug loading.

The term “acceptable dimensions and drug loading” as used herein encompasses a film with dimensions of up to three centimeters by five centimeters (length by with) and two millimeters of thickness and a drug loading ranging from 1.5%-60% of the total weight of the film.

The term “solid oral dosage form” as used herein encompasses a physical form of a predetermined amount of medication that may contain liquid or gaseous matter, but is primarily composed of solid matter having a higher Young's modulus and/or shear modulus than liquids.

The term “improved solubilization” as used herein encompasses Tadalafil with a degree of solubilization, at or below room temperature, of greater than 15 mg of Tadalafil per mL of liquid solvent(s) and preferably greater than 25 mg of Tadalafil per mL of liquid solvent(s) and more preferably greater than 30 mg per mL of liquid solvent(s).

The term “Tadalafil solubility enhancer” as used herein encompasses polyvinyl pyrrolidone, polyvinyl pyrrolidone derivatives, or another solid substance that when added to a solvent system containing one or more solvents capable of maintaining the Tadalafil solubility enhancer and Tadalafil in solution, provides improved solubilization of Tadalafil.

The term “plasticizer” as used to describe and claim certain embodiments of the invention encompasses a chemical entity that, when present, reduces the glass-transition temperature of amorphous polymers. A particular embodiment of the invention incorporates a plasticizer to impart flexibility, enhance elasticity and decrease brittleness. Preferred plasticizers include triacetine, citrate derivatives (such as triethyl, tributyl, acetyl tributyl, acetyl triethyl, trioctyl, acetyl trioctyl, trihexyl citrate, etc.) and dibutyl sebacate. Other embodiments of the invention do not include a plasticizer.

The term “penetration enhancer” as used herein encompasses a substance that can increase buccal permeation of an active ingredient and thereby enable a transcellular route for transportation of the drug through the buccal epithelium. Certain non-limiting examples of pharmaceutically acceptable penetration enhancers include benzalkonium chloride, cetylpyridinium chloride, cyclodextrins, dextran sulfate, lauric acid/propylene glycol, menthol, oleic acid, oleic acid derivatives, polyoxyethylene, polysorbates, sodium EDTA, sodium lauryl sulfate, sodium salicylate.

The term “therapeutically effective amount” refers to an amount of Tadalafil in a dosage form that becomes biologically available upon administration to exhibit clinically observable improvement of erectile dysfunction.

The term “surfactant” as used to describe and claim certain embodiments of the invention refers generally to a chemical compound or substance that, when present in an effective amount, reduces the surface tension of a liquid and the interfacial tension between liquids.

A process that produces a system that provides Tadalafil with improved solubilization comprises first dispersing, suspending and/or partially dissolving Tadalafil and optionally one or more antioxidants, one or more plasticizers, one or more colorants, one or more penetration enhancers and/or one or more optional surfactants in a solvent system containing at least one solvent, mixing until such time as all the ingredients capable of being dissolved are fully dissolved, then adding to the resulting solution a Tadalafil solubility enhancer. In certain embodiments, the Tadalafil solubility enhancer is added to a vortex at a mass sufficient to fully dissolve the Tadalafil without adding additional quantities of solvent. One or more other optional ingredients and/or other optional film forming polymers can be added to achieve desired properties. The mixture is then kept under rotation until the film forming polymers have completely dissolved and/or a homogenous blend has been obtained. Optional ingredients such as flavors, sweetener, taste-maskers, antioxidants and colorants can be added at any time. The addition of other optional, non active ingredients is completed at an appropriate time as to minimize potential segregation, physical-chemical incompatibility or partial dissolution of the film forming polymers.

Examples of suitable liquid solvents include, but are not limited to, alcohols, ketones, water, nitrile, chloroform, acetic acid, chlorinated solvents, aromatic solvents, hydroxylic solvents, and/or mixtures therefore, preferred liquid solvents include ketones, aliphatic alcohols and/or mixtures thereof and more preferably a mixture of acetone and methanol. Suitable solvents are solvents capable of dissolving the Tadalafil solubility enhancer and forming an environment that allows for improved solubilization of Tadalafil.

When producing a solid oral film dosage form, the final viscosity of the blend affects the film casting potential. Optimal viscosity ranges from 1000 centipoise to 90,000 centipoise. In certain embodiments of the invention, the final blend is transferred onto a surface of a suitable carrier material and dried to form a film. The carrier material must have a suitable surface tension in order to facilitate the homogenous distribution of the polymer solution across the intended coating width, without the formation of a destructive bond between the film and the carrier. Examples of suitable materials include non-siliconized polyethylene terephthalate film, non-siliconized paper, polyethylene-impregnated kraft paper, and non-siliconized polyethylene film. The transfer of the solution onto the carrier material can be performed using any conventional film coating equipment. A suitable coating technique would involve a knife-over-roll coating head. The thickness of the resulting film depends on the concentration of solids in the coating solution and on the gap of the coating head and can vary between 1 and 2000 μm. Drying of the film may be carried out in a high-temperature air-bath using a drying oven, drying tunnel, vacuum drier, or any other suitable drying equipment. A desired dry film thickness of about 70 μm is typically targeted to facilitate the administration, drying and processing of the film. However, it is possible to make thinner and thicker films.

The following examples illustrate methods of preparing formulations, oral film dosage forms and other oral dosage forms in accordance with certain non-limiting aspects of the invention. All percentages in the examples are by weight unless otherwise indicated.

### Example 1

The following example describes a process for preparing solid oral film dosage forms comprising Tadalafil for buccal and/or sublingual administration.

1.6 g of Tadalafil is dispensed in a solution comprised of 40.0 mL of acetone and 3 mL of methanol and containing 0.02 g of colorant Yellow # 5. To the resulting solution the Tadalafil solubility enhancer, polyvinyl pyrrolidone, is added slowly to a vortex at a mass required to complete the solubilization of the Tadalafil (1.0 to 5.0 g). To the resulting blend 0.03 g of sucralose, 1.0 g of triethyl citrate, 0.3 g of polysorbate 80 is added, and the mixture is stirred until homogenous. To the mixture, 1.0 g of hydroxypropyl cellulose is then added. The blend is stirred for 3 hours before adding and 0.2 g of vanilla flavor, mixed until homogenous, coated onto a suitable carrier material, and dried.

### Example 2

A mucoadhesive formulation was developed for preparing solid oral dosage forms for buccal and/or sublingual administration of a mixture containing Tadalafil.

From 1.5 g to 1.7 g of Tadalafil is dispensed in a solution containing 0.1 to 10 mL of methanol and 20.0 ml to 30.0 ml of acetone. To the resulting solution the Tadalafil solubility enhancer (polyvinyl pyrrolidone) is added slowly to a vortex at a mass required to precipitate the Tadalafil and the Tadalafil solubility enhancer (1.0 to 5.0 g). The resulting mixture is dried under vacuum.

The mixture is then added to other excipients to give the final formulation:

TABLE

| Ingredients | Function | (%) |
|---|---|---|
| Tadalafil—Tadalafil solubility | complex | 1.00-90.00 |
| enhancer mixture | | |
| Sodium Bicarbonate | effervescent | 0.00-10.00 |
| Menthol | taste masking agent | 0.00-10.00 |
| Sucralose | sweetener | 0.00-10.00 |
| Polyacrylic acid | mucoadhesive/hydrogel | 0.00-10.00 |
| Sorbitol | binder/filler | 0.00-50.00 |
| Mannitol | binder/filler | 0.00-50.00 |
| Isomalt sugar | binder/filler | 0.00-50.00 |
| Magnesium Stearate | lubricant | 0.00-0.50 |
| Poloxamer | solubility enhancer | 0.00-10.00 |
| Tablet weight and size | | 100 mg/7.0 mm |

### Example 3

In this example, a solid oral film dosage form comprising Tadalafil for buccal and/or sublingual administration is prepared without a surfactant.

1.0 to 1.2 g of Tadalafil is dispensed in a solution comprised of 30.0 mL of acetonitrile and 5.0 mL of methanol and containing 0.005 g of colorant Blue # 1. To the resulting solution the Tadalafil solubility enhancer, copovidone, is added slowly to a vortex at a mass required to complete the solubilization of the Tadalafil (1.0 to 7.0 g). To the resulting blend 0.03 g of sucralose are added and the mixture is stirred until homogenous. To the mixture, 2.0 g of hydroxypropyl cellulose is then added. The blend is stirred for 3 hours before adding and 0.2 g of vanilla flavor, mixed until homogenous, coated onto a suitable carrier material, and dried.

Modifications of the invention will occur to those skilled in the art and to those who make or use the invention. Therefore, it is understood that the embodiment(s) shown and described above are merely for illustrative purposes and not intended to limit the scope of the invention, which is defined by the following claims as interpreted according to the principles of patent law, including the doctrine of equivalents.

1. A pharmaceutical oral dosage form comprising Tadalafil prepared by:
dissolving a therapeutically effective amount of Tadalafil in a liquid medium containing one or more solvents and at least one Tadalafil solubility enhancer; and drying the mixture.

2. The dosage form of claim 1, further comprises one or more ingredient selected from hydroxypropyl cellulose, hydroxyethyl cellulose, or hydroxypropylmethyl cellulose, carboxymethyl cellulose. carbomers, pregelatinized modified starch, polyvinyl alcohol, sodium alginate, polyethylene glycol, natural gums like xanthane gum, tragacantha, guar gum, acacia gum, arabic gum or carboxyvinyl copolymers, polyethylene glycols, polyoxyl glycerides, propylene glycol esters, diethylene glycol esters, glyceryl esters, polyoxyethylene sorbitan fatty acid esters; ethylene alkyl ethers; polyoxyethylene alkyl phenols; polyethylene glycol glycerol fatty acid esters; polyoxyethylene-polyoxypropylene block copolymers; polyoxyethylene glycerides; polyoxyethylene sterols; polyoxyethylene vegetable oils; and polyoxyethylene hydrogenated vegetable oils.

3. The dosage form of claim 1, wherein said Tadalafil solubility enhancer is polyvinyl pyrrolidone.

4. The dosage form of claim 1, wherein said Tadalafil solubility enhancer is a solid substance that when added to a solvent system improves solubilization of the Tadalafil.

5. The dosage form of claim 1, further comprising a plasticizer selected from triethyl, tributyl, acetyl tributyl, acetyl triethyl, trioctyl, acetyl trioctyl, trihexyl citrate, dibutyl sebacate, triacetine, or derivatives thereof.

6. The dosage form of claim 1, further comprising at least one surfactant.

7. The dosage form of claim 1, further comprising at least one penetration enhancer selected from benzalkonium chloride, cetylpyridinium chloride, cyclodextrins, dextran sulfate, lauric acid/propylene glycol, menthol, oleic acid, oleic acid derivatives, polyoxyethylene, polysorbates, sodium EDTA, sodium lauryl sulfate or sodium salicylate.

8. The dosage form of claim 1, further comprising at least one sweetener.

9. The dosage form of claim 1, further comprising at least one colorant.

10. The dosage form of claim 1, further comprising at least one taste masker.

11. The dosage form of claim 1, further comprising at least one antioxidant.

12. The dosage form of claim 1, further comprising at least one flavoring agent.

13. The dosage form of claim 1, further comprising at least one mucoadhesive polymer.

14. The dosage form of claim 1, further comprising at least one pharmaceutically acceptable excipient.

15. A method of administering a therapeutically effective amount of Tadalafil, comprising:
disposing a therapeutically effective amount of Tadalafil into a liquid medium containing at least one Tadalafil solubility enhancer;
dissolving the Tadalafil;
casting and drying the composition to make a solid oral film form;
applying the dosage form in a subject's mouth and having the subject maintain the dosage form in the subject's mouth until an effective quantity of the dosage form has dissolved.

16. The method of claim 15, wherein is the liquid medium further comprises a plasticizer selected from triethyl, tributyl, acetyl tributyl, acetyl triethyl, trioctyl, acetyl trioctyl, trihexyl citrate, dibutyl sebacate, triacetine, or derivatives thereof.

17. The method of claim 15, wherein the liquid medium is non-polar.

18. The method of claim 15, wherein the liquid medium is polar.

19. The method of claim 15, wherein said Tadalafil solubility enhancer is polyvinyl pyrrolidone.

20. The method of claim 15, wherein said Tadalafil solubility enhancer is a solid substance that when added to a solvent system containing one or more solvents improves solubilization of the Tadalafil.

21. A method of producing a pharmaceutical oral film dosage form comprising:
dissolving a therapeutically effective amount of Tadalafil in a suitable liquid medium containing at least one Tadalafil solubility enhancer; and
casting and drying the composition to make a solid oral film.

22. The dosage form of claim 21, further comprising one or more ingredients selected from hydroxypropyl cellulose, hydroxyethyl cellulose, or hydroxypropylmethyl cellulose, carboxymethyl cellulose. carbomers, pregelatinized modified starch, polyvinyl alcohol, sodium alginate, polyethylene glycol, natural gums like xanthane gum, tragacantha, guar gum, acacia gum, arabic gum or carboxyvinyl copolymers, polyethylene glycols, polyoxyl glycerides, propylene glycol esters, diethylene glycol esters, glyceryl esters, polyoxyethylene sorbitan fatty acid esters; ethylene alkyl ethers; polyoxyethylene alkyl phenols; polyethylene glycol glycerol fatty acid esters; polyoxyethylene-polyoxypropylene block copolymers; polyoxyethylene glycerides; polyoxyethylene sterols; polyoxyethylene vegetable oils; and polyoxyethylene hydrogenated vegetable oils.

23. The dosage form of claim 21, further comprising one or more ingredients selected from polyethylene glycols, polyoxyl glycerides, propylene glycol esters, diethylen glycol esters, glyceryl esters, polyoxyethylene sorbitan fatty acid esters; ethylene alkyl ethers; polyoxyethylene alkyl phenols; polyethylene glycol glycerol fatty acid esters; polyoxyethylene-polyoxypropylene block copolymers; polyoxyethylene glycerides; polyoxyethylene sterols; polyoxyethylene vegetable oils; and polyoxyethylene hydrogenated vegetable oils.

24. The dosage form of claim 21, further comprising a plasticizer selected from triethyl, tributyl, acetyl tributyl, acetyl triethyl, trioctyl, acetyl trioctyl, trihexyl citrate, dibutyl sebacate, triacetine, or derivatives thereof.

25. The dosage form of claim 21, further comprising at least one surfactant.

26. The dosage form of claim 21, further comprising at least one penetration enhancer selected from benzalkonium chloride, cetylpyridinium chloride, cyclodextrins, dextran sulfate, lauric acid/propylene glycol, menthol, oleic acid, oleic acid derivatives, polyoxyethylene, polysorbates, sodium EDTA, sodium lauryl sulfate or sodium salicylate

27. The dosage form of claim 21, further comprising at least one sweetener.

28. The dosage form of claim 21, further comprising at least one colorant.

29. The dosage form of claim 21, further comprising at least one taste masker.

30. The dosage form of claim 21, further comprising at least one antioxidant.

31. The dosage form of claim 21, further comprising at least one flavoring agent.

32. The dosage form of claim 21, further comprising at least one mucoadhesive polymer.

33. The dosage form of claim 21, further comprising at least one pharmaceutically acceptable excipient.

34. The pharmaceutical oral dosage form of claim 1, in which the dried mixture is dispersed in or on a carrier material.
